(19) United States
(12) Patent Application Publication
(10) Pub. No.: US 2026/0204381 A1
(43) Pub. Date: Jul. 16, 2026

(54) METHOD OF OPERATION OF AN INFUSION PUMP FOR OPERATION IN A WIRELESS COMMUNICATION NETWORK, INFUSION PUMP AND COMMUNICATION MODULE

(71) Applicant: Fresenius Vial SAS, Brézins (FR)

(72) Inventors: Jean-Baptiste Audrain, Reaumont (FR); Vincent Chaboud, Doissin (FR)

(21) Appl. No.: 19/130,938

(22) PCT Filed: Feb. 21, 2024
(86) PCT No.: PCT/EP2024/054357

§ 371 Date: May 16, 2025

(30) Foreign Application Priority Data
Feb. 24, 2023 (EP) 23315040.8

(51) Int. Cl. G16H 20/17; G16H 40/67

(52) U.S. Cl. CPC G16H 20/17; G16H 40/67

(57) ABSTRACT

A method of operation of an infusion pump for operation in a wireless communication network is provided, wherein the method comprises: detection of a shutdown order by the infusion pump, wherein the shutdown order signals a switch off or a switch to standby mode of the infusion pump; transmission of a wake-up call from the infusion pump to a communication module in the wireless communication network in response to the detection of the shutdown order; switch off or switch to standby mode of the infusion pump, if a confirmation message was received from the communication module during a first timeout interval, wherein the confirmation message signals to the infusion pump that a data transmission to a remote server of the wireless communication network has been completed by the communication module; and transmission of a timeout-expiration message to the communication module, if no confirmation message was received from the communication module during the first timeout interval. A corresponding infusion pump and a communication module for operation in a wireless communication network as well as a system for operation in a wireless communication network are likewise provided.

The invention relates to a method of operation of an infusion pump for operation in a wireless communication network, as well as a method of operation of a communication module for operation in a wireless communication network, an infusion pump, a communication module and a system comprising an infusion pump and a communication module.

Infusion pumps administer medication and anesthesia to patients in an automated and controlled way. During infusion, the pump registers and/or measures information relating to the infusion. Such information comprises for example the infusion rate, intermissions (“stop” and “resume” events) and the type of medication administered. In order to enable remote patient monitoring and care, infusion information is regularly transmitted to a documentation server, also referred to as EMR server (EMR, electronic medical records). This automated, regular transmission of infusion information from the infusion pump to an EMR server, usually via a dedicated application server, is referred to as auto-documentation. The infusion information is stored on the EMR server as part of the electronic medical records. The EMR constitutes a local documentation system comprising medical data associated with the patients treated in the local unit, e.g. a hospital. The infusion information can then be displayed on an EMR user interface of a terminal or user device coupled with the EMR server. This enables remote access and manipulation of infusion information. For example, an infusion chart can be remotely displayed and signed off by a nurse or doctor. The interval at which each infusion pump transmits its infusion information or infusion status to the application server, and hence to the remote server and its documentation system, defines the auto-documentation cycle.

In order to enable wireless communication between the infusion pump and the application server, the infusion pump is connected to a communication module, e.g. via a wired LAN-connection. The communication module, also referred to as wifi module, is configured to wirelessly communicate with the application server, e.g. via a WLAN-connection. Hence, infusion pump, communication module and application server are part of a wireless communication network. In practice, multiple infusion pumps are connected to one communication module. Multiple infusion pumps are usually connected to one communication module via a rack. For example, multiple infusion pumps are stacked on top of each other in a rack of an infusion pump station. The pumps are interconnected with each other via the rack and the rack itself is connected with the communication module.

At the end of an infusion, there is the probability that the pump is immediately turned off. In this case, the last auto-documentation data are not transmitted to the application server, because the communication module, or wifi module, switches off upon turn-off of the pump. Hence, the application server is missing auto-documentation data. The auto-documentation data received by the application server are incomplete. The amount of missing data depends on the length of the auto-documentation cycle. The longer the cycle, the more data are potentially missing. In any case, the last event, namely the switch off event itself, is not recorded as part of the auto-documentation data.

It is hence an objective of the present invention to improve the handling of data, e.g. auto-documentation data, transmitted between an infusion pump and a remote server via a communication module, in particular with regard to data-loss protection and error management.

This objective is achieved by means of a method of operation of an infusion pump for operation in a wireless communication network, wherein the method comprises the steps of detecting a shutdown order by the infusion pump, wherein the shutdown order signals a switch off or a switch to standby mode of the infusion pump; transmitting a wake-up call from the infusion pump to a communication module in the wireless communication network in response to the detection of the shutdown order; switching off or switching to standby mode of the infusion pump, if a confirmation message was received from the communication module during a first timeout interval, wherein the confirmation message signals to the infusion pump that a data transmission to a remote server of the wireless communication network has been completed by the communication module; and transmitting a timeout-expiration message to the communication module, if no confirmation message was received from the communication module during the first timeout interval.

Hence, instead of immediately switching off or switching to standby when a shutdown order is detected, the infusion pump transmits a wake-up call to the communication module. This wake-up call triggers a last auto-documentation cycle during which the communication module tries to transmit missing auto-documentation data to the remote server. This last auto-documentation cycle hence does not share the same triggers as the normal auto-documentation cycle. It is exclusively triggered by the wake-up call. After the transmission of the wake-up call, the infusion pump waits a first timeout interval. Preferably, this first timeout interval is long enough to allow for a complete transmission of data of an average auto-documentation cycle. For example, the first timeout interval lasts less than 60 seconds, e.g. 30 seconds. If during that first timeout interval, the infusion pump receives a confirmation message from the communication module signaling that the data transmission to the remote server has been completed, hence, the last auto-documentation cycle has been successfully completed, it switches off or switches to standby, depending on the shutdown order. The first timeout interval provides additional time to the communication module during which it can try to effect a save and complete transmission of residual data to the remote server. If no confirmation message has been received during the first timeout interval, the infusion pump signals the expiration of the first timeout interval to the communication module with a timeout-expiration message. The present method thus improves the handling of auto-documentation data. With the present method, the likelihood that the auto-documentation data are complete at the remote server is increased. Further, the present method enables error tracking in case a complete data transmission cannot be accomplished.

The remote server is, in one example, a dedicated application server. That means, the remote server is configured to execute software associated with the handling of data for and from the infusion pump or multiple infusion pumps. The remote server is, for example, connected with a documentation or EMR server, e.g. of a hospital such that the remote server can transmit and receive data, in particular auto-documentation data received from or for an infusion pump, to the documentation or EMR server. The communication module is configured to enable wireless communication with other nodes in a wireless communication network, e.g. a wifi module. It is in communication connection with one or multiple infusion pumps, e.g. via a serial connection.

In one embodiment, the infusion pump switches off or switches to standby mode after expiry of a second timeout interval after transmission of the timeout-expiration message. For example, the second timeout interval is shorter than the first timeout interval. The second timeout interval is e.g. less than 10 seconds, for example 3 seconds. The second timeout interval provides additional time to the communication module in order to handle, e.g. store, information before it is being switched off or switched to standby as a consequence of a corresponding switch off or switch to standby of the infusion pump. E.g. the second timeout interval provides the communication module with time to store and/or transmit to the remote server information associated with the received timeout-expiration message.

In one embodiment, the transmission of the wake-up call to the communication module is triggered exclusively by the shutdown order. The wake-up call is therefore only initiated by a shutdown order. The wake-up call constitutes then the unique trigger for the last auto-documentation cycle. Only when the pump is switched off or switched to standby, a wake-up call is transmitted and a last auto-documentation cycle initiated.

The transmission of the wake-up call comprises the transmission of an electrical signal sent by the pump to the communication module, e.g. an internal electrical signal sent by the pump to the communication module using UART protocol, in particular a CTS signal. In one embodiment, the transmission of the wake-up call comprises the transmission of a status message comprising information on the status of the pump. In particular, information on whether the pump is about to switch off or switch to standby and/or information on whether the pump is on battery or on mains. For example, the status message is a C2h/B5h message, wherein the flags set in that message signal to the communication module whether the switch off or switch to standby is in progress with pump on battery or with pump on mains. Subidentifier B5 h signals read information. Other information may additionally or alternatively be transmitted in the status message.

In one embodiment, the wake-up call is repeated a defined number of times at defined intervals before the first timeout interval starts. This redundancy increases the reliability of the method. E.g. the wake-up call is repeated three times at time intervals with a minimum duration of 600 ms each, before the first timeout interval starts. That means, between each wake-up call, transmission is paused at least for 600 ms. In one embodiment, the transmission is paused for 600 ms. The first timeout interval starts only when transmission of the third wake-up call has been completed.

In one embodiment, the infusion pump receives a fail message from the communication module, wherein the fail message signals to the infusion pump that sending of residual data by the communication module has failed and wherein the infusion pump switches off or switches to standby after receipt of the fail message and before expiration of the first timeout interval. Therefore, the infusion pump does not unnecessarily wait for an expiration of the first timeout interval if the transmission of residual data from the communication module to the remote server is not possible. Instead, it switches off immediately.

In one embodiment, the infusion pump transmits an acknowledgement message to the communication module upon receipt of the confirmation message. The acknowledgement message is for example a C2h/35h command, wherein the flags signal the acknowledgement. In one embodiment, the confirmation message is a 42h/35h command and the infusion pump responds with the C2h/35h command as acknowledgement. Hence, command 42h is answered by a C2h command if successfully received, wherein subidentifier 35h signals write information.

A further object of the present invention is an infusion pump for operation in a wireless communication network, wherein the infusion pump is configured to detect a shutdown order, wherein the shutdown order signals a switch off or a switch to standby mode of the infusion pump; transmit a wake-up call to a communication module of the wireless communication network in response to the detection of the shutdown order; switch off or switch to standby mode of the infusion pump if a confirmation message was received from the communication module during a first timeout interval, wherein the confirmation message signals to the infusion pump that a data transfer has been completed by the communication module; and transmit a timeout-expiration message to the communication module, if no confirmation message was received from the communication module during the first timeout interval. The infusion pump improves the handling of auto-documentation data, in particular with regard to completeness of auto-documentation data on a remote server and also in case of a switch off or switch to standby of the infusion pump.

A further object of the present invention is a method of operation of a communication module configured for operation in a wireless communication network, wherein the method comprises receiving a wake-up call from an infusion pump in the wireless communication network; initiating the transmission of data to a remote server of the wireless communication network in response to the reception of the wake-up call; transmitting a confirmation message to the infusion pump if transmission of the data is completed and no timeout-expiration message has been received from the infusion pump until completion of the transmission of the data. This method improves the handling of data, in particular auto-documentation data, in particular with regard to their completeness on a remote server.

A further object of the present invention is a communication module for operation in a wireless communication network, wherein the communication module is configured to receive a wake-up call from an infusion pump in the wireless communication network; initiate the transmission of data to a remote server of the wireless communication network in reaction to the reception of the wake-up call; transmit a confirmation message to the infusion pump if transmission of the data is completed and no timeout-expiration message has been received from the infusion pump until completion of the transmission of the data. The communication module improves the handling of data, in particular auto-documentation data, in particular with regard to their completeness on a remote server, even in the event of a switch off or switch to standby of the connected infusion pump. The communication module is, for example, a network node of the wireless communication network. The communication module is e.g. a wifi module that enables wireless communication with other network nodes of the network. The communication module is configured to provide the wireless transmission of data, in particular auto-documentation data, from the infusion pump to the remote server.

Lastly, an object of the present invention is a system for operation in a wireless communication network, wherein the system comprises an infusion pump according to the invention and a communication module according to an embodiment of the present invention, wherein the infusion pump is communicatively coupled to the communication module. The infusion pump is, for example, coupled to the communication module via a bus interface.

Further, a method of operation in a system according to claim 14 is disclosed, which comprises the steps of: detecting a shutdown order by an infusion pump, wherein the shutdown order signals a switch off or a switch to standby mode of the infusion pump; transmitting a wake-up call by the infusion pump to a communication module of the wireless communication network in response to the detection of the shutdown order; receiving the wake-up call from the infusion pump by the communication module; initiating the transmission of data to a remote server of a wireless communication network by the communication module in reaction to the reception of the wake-up call; transmitting a confirmation message by the communication module to the infusion pump if the transmission of data is completed by the communication module and no timeout-expiration message has been received by the communication module from the infusion pump until completion of the transmission of data; switching off or switching to standby mode of the infusion pump if a confirmation message was received by the infusion pump from the communication module during a first timeout interval, wherein the confirmation message signals to the infusion pump that a data transfer has been completed by the communication module; and transmitting a timeout-expiration message to the communication module, if no confirmation message was received from the communication module during the first timeout interval. In one embodiment, initiating transmission of data by the communication module to the remote server is prevented if a battery alarm occurs on the infusion pump and the occurrence of this battery alarm is known to the communication module, e.g. because it has requested this information from the infusion pump.

The idea underlying the invention shall subsequently be described in more details with respect to the embodiments shown in the drawings. For the sake of legibility and in order to avoid repetitions, the description of the drawings focusses on the claimed methods. They do, however, likewise illustrate the infusion pump, the communication module and the system according to embodiments of the present invention. Herein:

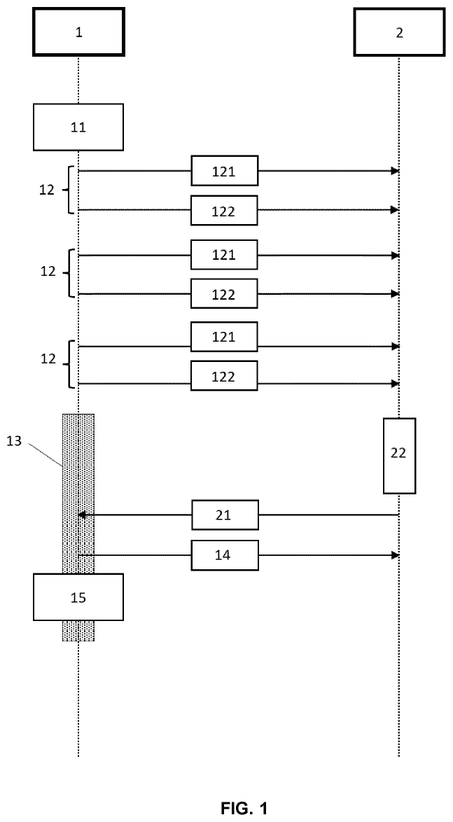

FIG. 1 shows a method of operation in an infusion pump for operation in a wireless communication network as well as a method of operation in a communication module according to one embodiment of the present invention;

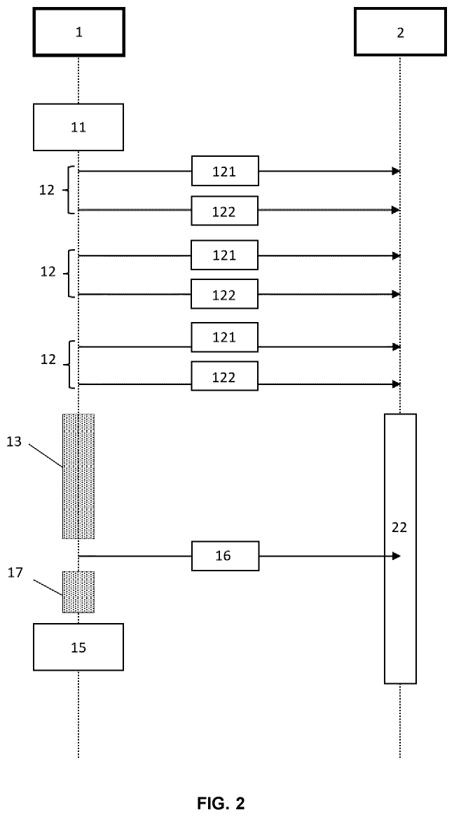

FIG. 2 shows a method of operation in an infusion pump for operation in a wireless communication network as well as a method of operation in a communication module according to an embodiment of the present invention.

FIG. 1 shows a method of operation in an infusion pump 1 for operation in a wireless communication network as well as a method of operation in a communication module 2 according to one embodiment of the present invention. The infusion pump 1 and the communication module 2 are in communication connection. For example, they are connected via a serial interface, e.g. a serial bus. Multiple infusion pumps 1 may be connected to one communication module 2. The communication module 2 is, in one embodiment, configured to be connected to multiple infusion pumps 1. The infusion pump 1 and the communication module 2 form a system according to an embodiment of the present invention. The infusion pump 1 receives a shutdown order 11, e.g. initiated by the pressing of a corresponding button on the infusion pump 1. The shutdown order 11 signals a switch off or switch to standby to the infusion pump 1. For example, the extent of time during which the button is pressed on the pump 1 signals whether the infusion pump 1 shall switch off or switch to standby. The infusion pump 1 detects the shutdown order 11 and, as a consequence, transmits a wake-up call 12 to the communication module 2. In the present embodiment, the wake-up call is repeated a defined number of times, e.g. three times, with intermittent time intervals during which transmission is paused. For example, the intermittent time intervals are at least 600 ms each. Each wake-up call 12 comprises an electrical signal 121, in particular a CTS signal, sent by the pump 1 to communication module 2. Additionally, the wake-up call 12 may comprise a status message 122 carrying information on the pump status. Such pump status information may be information on whether the pump 1 is about to switch off or about to switch to standby. Alternatively or additionally, the pump status information may be information on whether the pump 1 was on battery or on mains when the shutdown order 11 was detected or initiated. The status message 122 is e.g. a C2h/B5 h command. Depending on whether the pump 1 is on battery or on mains upon detection of the shutdown order 11, the flags are set to 0x01 or 0x02. This is a particular efficient way of signaling to the communication module 2 whether the pump 1 is on battery or on mains. After transmission of the last wake-up call, a first timeout interval 13 starts on the infusion pump 1. During the first timeout interval 13, the pump 1 waits for a response from the communication module 2. The first timeout interval 13 is e.g. 30 seconds. Upon reception of one of the wake-up calls 12, the communication module 2 starts a last auto-documentation cycle 22. That means, the communication module 2 starts transmission of residual data that have not yet been transmitted to the remote server. If the transmission of residual data, i.e. the last auto-documentation cycle 22, is completed successfully during the first timeout interval 13, the communication module 2 transmits a confirmation message 21 to the pump 1. If the pump 1 receives this confirmation message 21 in the first timeout interval 13, it responds with an acknowledgement message 14. For example, the confirmation message 21 is a 42h/35h command with flags set to 0x00 and the acknowledgement message is a C2h/35h command with flags set to 0x00 as well. In this way, pump 1 and communication module 2 assert each other of a successful data transmission to the remote server and the reception of this information on the pump 1 side. As a confirmation message 21 has been received by the pump 1, the pump 1 switches off or switches to standby 15, respectively, after sending of the corresponding acknowledgement message 14. The provision of the first timeout interval 13 together with the transmission of the wake-up call 12 to the communication module 2 ensures the completeness of the data transfer from the infusion pump 1 to a remote server via the communication module 2. Hence, data handling, in particular of auto-documentation data, is improved.

In case a battery alarm occurs on the pump 1 before the communication module 2 starts the sending of residual data, i.e. the last auto-documentation cycle, the transmission of residual data is not initiated. That means, in case of a battery alarm, not last auto-documentation cycle takes place. In this case, the pump 1 will switch off or switch to standby 15, respectively, without starting the first timeout interval 13. In particular, the pump 1 will switch off or switch to standby in response to the detection of a shutdown order, e.g. if a corresponding button is pressed on the pump 1, immediately. Thus, pump autonomy on battery is preserved.

In case the communication module 2 is not able to transmit the residual data, i.e. perform the last auto-documentation cycle, it transmits a corresponding fail message to the pump 1. The fail message signals to the pump 1 that sending of residual data has failed. The pump 1 then switches off or switches to standby after receipt of this message and before expiration of the first timeout interval 13. Hence, an unnecessary elapse of time between initiation of the shutdown order, e.g. through pressing of a corresponding button on the pump 1, and the actual switch off or switch to standby is avoided.

FIG. 2 shows a method of operation in an infusion pump 1 for operation in a wireless communication network as well as a method of operation in a communication module 2 according to an embodiment of the present invention. As detailed with regard to FIG. 1, the pump 1 transmits a wake-up call 12, or multiple wake-up calls 12, to the communication module 2 upon detection of a shutdown order 11. After the wake-up call 12 is completed, the first timeout interval 13 starts on the pump 1. Whereas FIG. 1 illustrates the case, wherein the confirmation message 21 from the communication module 2 is received by the pump 1 in the first timeout interval 13, FIG. 2 now illustrates the case, wherein no confirmation message 21 is received during the first timeout interval 13. If the first timeout interval 13 expires on the pump 1 before a confirmation message 21 is received from the communication module 2, the pump 1 transmits a timeout-expiration message 16 to the communication module 2. This timeout-expiration message 16 warns the communication module 2 that the first timeout interval 13 has expired. The timeout-expiration message 16 is e.g. a C2h/B5 h command with flags set to 0x04. In one embodiment, a second timeout interval 17 starts on the infusion pump 1 that is shorter than the first timeout interval 13. The second timeout interval 17 lasts e.g. 3 seconds. Only after expiration of this second timeout interval 17, does the pump 1 switch off or switch to standby mode 15, respectively. The timeout-expiration message 16 provides a form of error management and hence improves data handling.

### LIST OF REFERENCE NUMERALS

1 Infusion pump
2 Communication module
11 Shutdown order
12 Wake-up call
121 electrical signal sent by the pump to the communication module (CTS signal)
122 Status message
13 First timeout interval
14 Acknowledgement message
Switch off or switch to standby
16 Timeout-expiration message
17 Second timeout interval
21 Confirmation message
22 Auto-documentation cycle

1. A method of operation of an infusion pump for operation in a wireless communication network, the method comprising:
detecting a shutdown order by the infusion pump, wherein the shutdown order signals a switch off or a switch to standby mode of the infusion pump;
transmitting a wake-up call from the infusion pump to a communication module in the wireless communication network in response to the detection of the shutdown order;
switching off or switching to standby mode the infusion pump, if a confirmation message was received from the communication module during a first timeout interval, wherein the confirmation message signals to the infusion pump that a data transmission to a remote server of the wireless communication network has been completed by the communication module; and
transmitting a timeout-expiration message to the communication module, if no confirmation message was received from the communication module during the first timeout interval.

2. The method according to claim 1, wherein the infusion pump switches off or switches to standby mode after expiry of a second timeout interval after transmission of the timeout-expiration message.

3. The method according to claim 1, wherein the transmission of the wake-up call to the communication module is triggered exclusively by the shutdown order.

4. The method according to claim 1, wherein the transmission of the wake-up call comprises the transmission of an electrical signal sent by the pump to the communication module, wherein the electrical signal is a CTS signal.

5. The method according to claim 1, wherein the transmission of the wake-up call comprises the transmission of a status message comprising information on a status of the infusion pump.

6. The method according to claim 1, wherein transmitting the wake-up call is repeated a defined number of times at defined intervals, before the first timeout interval starts.

7. The method according to claim 6, wherein transmitting the wake-up call is repeated three times at time intervals with a minimum duration of 600 ms each, before the first timeout interval starts.

8. The method according to claim 1, wherein the infusion pump receives a fail message from the communication module, wherein the fail message signals to the infusion pump that sending of residual data by the communication module has failed and wherein the infusion pump switches off or switches to standby after receipt of the fail message and before expiration of the first timeout interval.

9. The method according to claim 1, wherein the infusion pump transmits an acknowledgement message to the communication module upon receipt of the confirmation message.

10. An infusion pump for operation in a wireless communication network, the infusion pump being configured to:
detect a shutdown order, wherein the shutdown order signals a switch off or a switch to standby mode of the infusion pump;
transmit a wake-up call to a communication module of the wireless communication network in response to the detection of the shutdown order;
switch off or switch to standby mode of the infusion pump if a confirmation message was received from the communication module during a first timeout interval, wherein the confirmation message signals to the infusion pump that a data transmission has been completed by the communication module; and
transmit a timeout-expiration message to the communication module, if no confirmation message was received from the communication module during the first timeout interval.

11. A method of operation of a communication module configured for operation in a wireless communication network, the method comprising:
receiving a wake-up call from an infusion pump in the wireless communication network;
initiating the transmission of data to a remote server of the wireless communication network in response to the reception of the wake-up call; and
transmitting a confirmation message to the infusion pump if transmission of the data is completed and no timeout-expiration message has been received from the infusion pump until completion of the transmission of the data.

12. A communication module for operation in a wireless communication network, wherein the communication module is configured to:
receive a wake-up call from an infusion pump in the wireless communication network;
initiate the transmission of data to a remote server of the wireless communication network in response to the reception of the wake-up call; and
transmit a confirmation message to the infusion pump if transmission of the data is completed and no timeout-expiration message has been received from the infusion pump until completion of the transmission of the data.

13. The communication module according to claim 12, wherein the communication module is a network node of the wireless communication network.

14. A system for operation in a wireless communication network, wherein the system comprises:
an infusion pump according to claim 10; and
a communication module configured to receive a wake-up call from an infusion pump in the wireless communication network, initiate the transmission of data to a remote server of the wireless communication network in response to the reception of the wake-up call, and transmit a confirmation message to the infusion pump if transmission of the data is completed and no timeout-expiration message has been received from the infusion pump until completion of the transmission of the data,
wherein the infusion pump is communicatively coupled to the communication module.

15. The system according to claim 14, wherein the infusion pump is coupled to the communication module via a bus interface.
